(19) United States
(12) Patent Application Publication
(10) Pub. No.: US 2011/0098462 A1
(43) Pub. Date: Apr. 28, 2011

(54) METHOD AND KIT FOR THE SAMPLE PRESERVATION AND CELL DISRUPTION BEFORE THE EXTRACTION OF NUCLEIC ACIDS

(75) Inventors: Rudolf Ehwald, Berlin (DE); Dietmar Lerche, Berlin (DE); Holger Woehlecke, Berlin (DE); Christina Lerche, Magdeburg (DE)

(73) Assignee: Dr. Lerche KG, Berlin (DE)

(21) Appl. No.: 12/809,468

(22) PCT Filed: Dec. 22, 2008
(86) PCT No.: PCT/EP08/68210

§ 371 Date: Jan. 12, 2011

(30) Foreign Application Priority Data
Dec. 21, 2007 (DE) 10 2007 063 019.2

(51) Int. Cl. C07H 21/00; C12N 5/02; C12M 1/00

(52) U.S. Cl. 536/25.42; 435/379; 435/283.1

(57) ABSTRACT

The invention relates to a method for preserving samples and disrupting cells which can be employed for the preparation of the extraction of nucleic acids from live cells, cell aggregates and tissue samples, and to a kit for carrying out the method. The method includes the introduction of live cells, cell aggregates or tissue samples whose nucleic acids are to be extracted into an excess of water-miscible and volatile organic liquid. The saturation of the sample with the organic liquid has an advantageous effect on the extraction of the nucleic acids, which is subsequently carried out in an aqueous medium. The mechanical cell disruption is facilitated by the virtually complete removal of the water from the cells. The biomembranes are dissolved in the organic liquid. tRNA and other RNA fractions with a low degree of polymerization, which are capable of permeating across the cell wall, can be extracted selectively without mechanical disruption. Before high-molecular-weight nucleic acids are extracted, the cells, cell aggregates or tissue samples are disrupted in the organic liquid with the aid of mechanical pulses. Since the nucleases are inactive in the dehydrated organic liquid, the biological samples or the homogenates prepared therefrom can be stored at room temperature in the organic liquid as long as desired. After incubation in the largely anhydrous organic liquid, the sample's nucleases are largely denatured. An advantageous kit according to the invention contains a mixed organic/aqueous phase with denaturing properties and, for each sample, a shaped body for dehydration which is adapted to suit the size of the preservation container, and a mixture, adapted to suit the size of the preservation container, of a few large and many small disruption bodies for the disruption in the nonaqueous medium. A considerable advantage of the invention is the fact that cooling and powerful mechanical pulses when disrupting the cells and during the extraction may be dispensed with, and that DNA extraction and cell disruption can be carried out at different times.

The invention relates to a method for preserving samples and disrupting cells which can be employed for the preparation of the extraction of nucleic acids from live cells, cell aggregates and tissue samples, and to a kit for carrying out the method. The method includes the introduction of live cells, cell aggregates or tissue samples whose nucleic acids are to be extracted into an excess of water-miscible and volatile organic liquid. The saturation of the sample with the organic liquid has an advantageous effect on the extraction of the nucleic acids, which is subsequently carried out in an aqueous medium. The mechanical cell disruption is facilitated by the virtually complete removal of the water from the cells. The biomembranes are dissolved in the organic liquid. tRNA and other RNA fractions with a low degree of polymerization, which are capable of permeating across the cell wall, can be extracted selectively without mechanical disruption. Before high-molecular-weight nucleic acids are extracted, the cells, cell aggregates or tissue samples are disrupted in the organic liquid with the aid of mechanical pulses. Since the nucleases are inactive in the dehydrated organic liquid, the biological samples or the homogenates prepared therefrom can be stored at room temperature in the organic liquid as long as desired. After incubation in the largely anhydrous organic liquid, the sample's nucleases are largely denatured. An advantageous kit according to the invention contains a mixed organic/aqueous phase with denaturing properties and, for each sample, a shaped body for dehydration which is adapted to suit the size of the preservation container, and a mixture, adapted to suit the size of the preservation container, of a few large and many small disruption bodies for the disruption in the nonaqueous medium. A considerable advantage of the invention is the fact that cooling and powerful mechanical pulses when disrupting the cells and during the extraction may be dispensed with, and that DNA extraction and cell disruption can be carried out at different times.

### DESCRIPTION OF THE INVENTION

It is essential for numerous applications in molecular biology to prevent the enzymatic degradation of the nucleic acids during preparation of the cell-free extract. Especially bacterial and fungal cells as well as plant cells have to be disrupted, since the cell walls are impermeable to the nucleic acids of higher molecular weight. The procedure mostly applied at present consists in the mechanical grinding of cells, cell aggregates and tissue samples that have been frozen in liquid nitrogen, and their subsequent transfer into an aqueous buffer used for extraction and solubilisation. The energy input for mechanical disruption brings about the danger of local thawing and possible action of nucleases. Additionally, high mechanical stress acting on the nucleic acids in the aqueous liquid phase can result in depolymerisation. Fragmentation of nucleic acids can also result from the growth of ice crystals when nucleic acids are preserved by freezing. The technical effort for preservation by freezing and cell disruption in liquid nitrogen renders sampling in the field difficult and causes expenses at the transport of the samples. RNA of high molecular weight is especially sensitive to enzymatic degradation. Therefore, analysis of transcription in living cells often requires especially complex protocols and high effort. It is essential that the nucleases in the cytosol, lysosomes, vacuols and cell walls are rapidly inactivated. If the samples contain RNAse with high activity, it requires the usual combination of denaturing additives to the extraction buffer, removal of calcium ions, low temperature and short extraction time, but even so results often in low quality of the RNA or nonsufficient yield. A procedure and medium for in situ RNA preservation in biological samples (U.S. Pat. No. 6,204,375) is based on the denaturing, dehydrating and precipitating action of concentrated salt solutions on nucleases and enables the later extraction of non-destructed RNA of high quality. The high salt concentration, however, can result in disadvantages for the subsequent investigations. Also preservation steps with concentrated salt solutions according to U.S. Pat. No. 6,204,375 do not avoid the problems connected with cell disruption. There remains a need for procedures suitable for sample preservation and cell disruption enabling one to extract high molecular weight nucleic acids from cells with rigid wall without loss of quality.

The invention aims to supply a technical procedure and a corresponding preparation kit to prepare biological samples from living cells, cell aggregates, and tissue particles for extraction of high-molecular weight DNA or RNA. The aimed procedure should include in situ preservation and cell disruption, not require a high input of mechanical energy and be efficient at room temperature without enzymatic or mechanical degradation of the nucleic acids. It should enable complete disruption of bacterial, fungal and plant materials to subcellular particles with dissolvable nucleic acids which can be subsequently extracted with high quality and high yield.

A procedure with the aimed advantages is described in claim 1 and a kit for sample preparation facilitating this procedure is described in claim 7. Further claims relate to favourable variants of the procedure and the kit.

The invention is based on our findings that yeast and bacterial cells as well as plant tissues, which are highly resistant to mechanical disruption, can be completely disrupted with low mechanical power in short time after their almost complete dehydration in an volatile organic liquid as acetone or ethanol, which is miscible with water. The water content of the organic liquid has to be below the water content of the azeotropic mixture of the respective organic liquid with water, e.g. in case of ethanol below 4% as the azeotropic mixture contains 96% ethanol.

In order to destroy all cell walls, it is for instance sufficient to shake an almost water-free yeast suspension in acetone with fine glass beads and some large glass beads (3 mm) on a conventional vortex mixer designed for mixing of small liquid volumes. In contrast, there was no significant cell disruption at the same mechanical treatment, if the glass beads and yeast cells were dispersed in the aqueous milieu. The efficiency of the cell disruption in the organic liquid was strongly dependent on the degree of removal of residual water from the cells and the organic medium. A relatively small amount of water in the organic liquid resulting from addition of 50 μl of an aqueous yeast suspension to 1.2 ml of pure acetone or ethanol, affected strongly the efficiency of cell disruption. It is therefore essential for the invention to remove residual water almost completely from the suspension of the cells, cell aggregates or tissue samples, this way preventing residual swelling of the hydrophilic polymers of the cell and especially of the cell walls with water, before samples are mechanically disrupted in the organic liquid. Generally, it was found that cells, cell aggregates and tissue samples got highly brittle only in the almost completely dehydrated state, thus enabling efficient mechanical disruption with relatively weak mechanical impulses. With a comparable effect it was possible to use other organic liquids that are miscible with water instead of acetone, for example methanol, ethanol, isopropanol and dioxane to name a few.

The invention, as other procedures for mechanical sample and cell disruption, requires a generator of mechanical impulses in a liquid volume, for instance a shaking or stirring device, a manual mortar or a source of ultrasound. It is essential for the invention that the mechanical impulses act on a biological material that is equilibrated with an almost water-free volatile organic liquid which is miscible with water.

A favourable variant of the procedure according to the invention consists in the addition of suitable cell disruption beads into the vessel that has been applied for fixation and preservation of the biological sample by the organic liquid and for its complete dehydration. For this purpose a suspension of cells, cell aggregates or tissue samples is prepared in a volume of the volatile hydrophilic organic liquid comprising preferably about 40 to 60% of the vessel volume. The vessels contain cell disruption beads made for instance of glass with a portion of 5 to 30% of the vessel volume. Preferably cell disruption beads consist of a mixture of fine glass beads with a diameter below 0.5 mm and a few large, heavy beads with a diameter of 2 to 5 mm, consisting for instance of glass or steel. The movement of the larger beads amplifies the convectional gradients in the cell disruption vessel. The high concentration of fine cell disruption beads brings about a high frequency of cell wall breaking mechanical impulses acting on the dehydrated cells, cell aggregates or tissue samples. The cell disruption beads are applied in the almost water-free medium in similar manner as it is usual in the aqueous milieu. The invented procedure using an almost water-free milieu has the advantage that the cell walls can be broken completely with only a low input of energy. The cells or tissue particles burst already under the influence of low mechanical impulses since the cell walls loose their ability of elastic deformation due to almost complete dehydration. A further advantage of the procedure according to the invention is based on the action of the mechanical impulses and shearing forces in the almost complete absence of the solvating water from the cells or tissue samples and the high-molecular-weight nucleic acids in the denatured cells are existent in a strongly condensed conformation and are therefore protected against depolymerisation by mechanical stress. This removes the danger of DNA degradation by shearing during the disruption step.

In the simplest case it is possible to dehydrate cells, cell aggregates and tissue samples almost completely by repeated extraction with the pure hydrophilic organic liquid, whereby the organic medium containing residual water is exchanged with a fresh water-free organic liquid. If the biological material contains a high endogenous activity of nucleases, phenol-oxidases or other oxidizing enzymes creating free radicals as usual for plant and fungal samples, it is possible that non-desired reactions of the nucleic acids with these enzymes take place during fixation in the organic liquid. For instance, it is possible that the extraction of the nucleic acids is prevented by the formation of polyphenol compounds and oxidative cross-linking of proteins. Therefore, in a variant of the invention the respective enzymatic processes are suppressed by mixing a buffer with low pH (pH 2 to 4) to the organic liquid at a volume ratio of 5 to 30%. This mixture is exchanged for the almost water-free organic liquid after a short period of its action (preferably 30 to 120 min). In addition, the mixture may contain a reducing substance as for instance sodium sulphite, sodium dithionite, or mercaptoethanol in order to strengthen the inhibititory effect of the low pH on the non-desired oxidation processes. Fixation of the biological sample in an organic liquid with a water content of ca. 20% before the successive exchange against the almost water-free medium has a further advantage. It is known that the presence of water in an organic liquid used for extraction of plant materials results in a more rapid and more complete dissolution of amphiphilic compounds such as phenols and to a more rapid extraction of lipids like phospholipids, carotene and chlorophyll.

According to the invention dehydration of the sample can be also carried out by addition of a solid substance suitable for dehydration of organic liquids to the preparation vessel with the sample and the organic liquid. Especially suitable for this purpose is zeolite (molecular sieve) with a pore size of 0.3 nm, which is used for the technical dehydration of alcohols and other organic liquids. Water-free crystals of sodium sulphate, magnesium sulphate and similar crystals are likewise suitable for this purpose. The dehydrating solid substance must be added in sufficient amount. The product of its amount and of the water binding capacity should be larger than the water content of the sample. Once the dehydrating substance is added in sufficient amount, the residual water is removed from the biological material without medium exchange.

An advantage of the invented procedure and the invented preparation kit can be seen in the fact that organic hydrophilic and volatile liquids like acetone, dioxane, methanol, ethanol and iso-propanol destroy the lipid membranes and penetrate rapidly into the cells or tissue particles, which get dehydrated, whereby nucleases and oxidizing enzymes are inactivated. During the incubation of the cells, cell aggregates and tissue samples in the water-free organic liquids the mentioned enzymes are also denatured irreversibly. Therefore it is possible to keep the nucleic acids without storage changes for long time. The tissue samples or cells, which are saturated with the organic liquid or the homogenates obtained by their mechanical disruption in the organic liquid, may be stored a long time without danger of enzymatic change even at room temperature. Since endogenous nucleases and oxidases can be denatured by the almost complete dehydration in the water-free organic liquid, in many cases the following extraction of the nucleic acids can be carried out at room temperature.

The organic liquid can be separated easily from the particulate cell debris, which contain the nucleic acids in non-dissolved state. The large difference in density and very small solvation of the hydrophilic colloids in the organic liquid brings about rapid and complete sedimentation of the dehydrated cell debris at centrifugation with low speed. The volatility of the organic liquid enables its rapid removal from the pellet by evaporation. If e.g. 2-ml-tubes (Eppendorf-tubes) are used as vessels for sample preservation and cell disruption the mobile part of the organic liquid is decanted or sucked off, whereas the residual liquid is removed completely during incubation of the open vessel at room temperature, by application of vacuum or by its warming up in a heating block. Since the organic liquid is almost completely dehydrated there is no danger of phase separation of residual water at this step of the procedure. Therefore, the hydrophilic cell fragments are not glued together by transient solvation with water and cohesive sticking at the end of the drying process. The resulting dry powder of cell debris can be equilibrated rapidly with the extraction buffer. Surprisingly it has been found that the solubility of the nucleic acids was not reduced by complete evaporation of the water-free volatile organic liquid from the cell debris.

In order to yield a clear aqueous crude extract after the dissolution of the nucleic acids it is necessary to remove non-dissolvable particles like fine cell wall fragments by micro-filtration or centrifugation. This step is not necessary, if a chromatographic column is used for the isolation of the nucleic acids. In this case the applied column can be covered by a micro-filter to remove interfering particles, when the crude extract flows into the chromatographic bed.

It is favourable to apply a preparation kit for the invented procedure. For each sample the invented preparation kit includes a solid almost completely water-free substance for binding of the water present in organic liquid. The dehydrating substance has to be protected against water uptake from the air by filling it in a water-tight vessel or envelope. It is especially favourable to use the dehydrating substance as a solid body adapted in shape and mass to the vessel in a way that it can be removed conveniently from the organic liquid after its complete dehydration. After removal of the dehydrating substance disruption beads may be added to the organic liquid with the biological sample. Now the preparation vessel with the sample and the disruption beads can be shaken to disrupt the tissue to cell debris.

Hence, in a preferred variant of the invention the preparation kit contains an amount of disruption beads or bodies that are adapted to the vessel volume, preferably a mixture of large and heavy particles, which create a strong convection in the organic liquid during shaking, and of smaller particles, which accelerate the disruption of small cells and tissue particles by a high density of mechanical impulses. The invention is elucidated by the following examples.

### EXEMPLARY EMBODIMENTS

### Example 1

Acetone was used as organic liquid. It was stored in a flask above dehydrated sodium sulphate. Samples of 50 μl of a concentrated yeast suspension (Saccharomyces cerevisiae) obtained by mixing a pellet of the centrifuged yeast suspension with the volume ratio 1:1 were added to closable preservation vessels (2 ml-Eppendorf tube) containing 1.2 ml of dehydrated acetone and 200 mg sodium sulphate. 50 μl of the yeast suspension were also added to control vessels containing 1.2 ml water-free acetone without sodium sulphate crystals. The vessels were tightly closed and incubated at room temperature with occasional shaking during the first hours.

After 24 hours or 8 weeks cell disruption was performed. For this purpose the dehydrated cell suspension was decanted from the crystals and transferred into a closable cell disruption vessel containing 200 mg of fine glass beads with a diameter of 1 to 10 μm (Spheriglass, Potter Europe, Suffolk, UK) together with 10 glass beads with a diameter of ca. 2 mm. The cell disruption vessels (Eppendorf tubes) were shaken on a vortex mixer. It was found by light microscopy that the cells were completely disrupted after 30 min, whereas disruption was incomplete of the cells of control samples which were not dehydrated by sodium sulphate. Cells pre-incubated in water did not show significant cell disruption. The tubes were centrifuged and the supernatant removed. The residual acetone was evaporated by vacuum.

The obtained dry powder was shaken with 0.4 ml of an aqueous extraction buffer (10 mM Tris, 10 mM EDTA, pH 8, 1.4% sodium dodecylsulphate) for 10 min and the suspension was subsequently incubated at 37° C. Nucleic acids of high molecular weight were separated from the dispersions with a column for isolation of nucleic acids according to DE 199 02 724 and DE 102004030878. In these columns there was a 3 mm thick filter layer consisting of fine particles (diameter 5 to 10 μm) on the chromatographic bed consisting of sporopollenin-microcapsules (1 ml). The elution was carried out with Na-EDTA solution (10 mM, pH 8), which was also applied for pre-equilibration of the column. DNA and RNA of high molecular weight were eluted with a volume from 0 to 0.37 ml and concentrated in the collecting fibre at the column outlet. There was collected a fraction of high-molecular weight DNA forming a sharp band with a size far above 10 000 by and high molecular weight RNA, as shown by agarose gel electrophoresis (0.8% agarose). Most of the obtained RNA was concentrated in the bands of the 28 S rRNA and that of the 18 S rRNA. The good quality of the RNA preparation was documented by the higher amount found in the 28 S band. It was remarkable that the high molecular nucleic acids remained intact even at a long period of extraction (up to 24 hours) at 37° C. The duration of the preservation period (24 h or 8 weeks) did not affect the sharpness and position of the bands. No decomposition of nucleic acids was detectable by agarose gel electrophoresis. It can be deduced that the invented procedure in this case denatured the nucleases irreversibly. This enabled prolonged extraction times without risk of enzymatic degradation at room temperature and even higher temperature and, therefore, a high yield of DNA and high-molecular weight RNA.

### Example 2

Ethanol was applied as organic liquid. A yeast suspension was prepared by mixing the pellet of a culture of Saccharomyces cerevisiae with water in the mass ratio 1:1. 50 μl samples of this suspension were suspended in 1 ml of 96% ethanol in 2 ml reaction vessels. One part of the sample (variant A) was shaken over night and thereafter the cells were collected by centrifugation. A further part of the sample (variant B) was centrifuged after four hours of shaking, then the pellet was suspended in 1 ml of absolute ethanol and then further shaken overnight. In a further sample (variant C) the water containing ethanol was likewise exchanged against absolute ethanol. In this variant a water absorbing strip was added to the suspension for complete removal of water from the suspension and removed after a dehydration period of approximately. 18 h.

The water absorbing strips were produced by gluing twenty zeolite beads with a diameter of 3 mm and a pore size of 0.3 nm on a 1.8 cm long and 4 mm wide strip of filter paper. The dehydration strips were dried in the vacuum (0.07 bar) of a freeze dryer at 80° C. and then enclosed in Eppendorf tubes before until use.

A mixture of cell disruption beads consisting of 10 glass beads with a diameter of 3 mm and 100 mg glass beads with diameter of 0.2 to 0.5 mm were added to the ethanolic yeast suspensions according to variants B and C. For cell disruption the tubes were shaken for 30 min on a vortex mixer with a frequency of 50 s⁻¹ and a shaking radius of 2.5 mm. The suspensions according to variant A were not treated for cell disruption by shaking with glass beads.

To extract the nucleic acids 0.1 ml of the ethanol suspension of the cells (variant A) and cell debris (variant B and C) were centrifuged in a 0.5 ml Eppendorf-tube. The ethanol supernatant was decanted and the pellet dried by evaporation. The pellets were shaken in 0.1 ml of an aqueous extraction buffer (1.4% SDS, 50 mM EDTA-Na, pH 7.4) at room temperature for 18 hours. The extracts were centrifuged and this way separated from the insoluble cell debris or cells. 10 μl of the extracts were investigated by electrophoresis on agarose gel (1.3%). In the electropherograms of variants B and C a sharp band of DNA with high molecular weight appeared and the RNA showed its highest concentration in the two bands of rRNA (28 S and 18 S rRNA). These bands were more prominent in the extracts of variant C (complete dehydration by help of the Zeolith) than in the case of variant B. Low-molecular-weight RNA with a size of about 80 nucleotides (mainly tRNA) was found in extracts of all variants. In the case of variant A which has not been treated for cell disruption, the extracts did not contain detectable amounts of high-molecular weight nucleic acids, but they contained low-molecular weight RNA (mainly tRNA) in comparable amount as variants B and C.

The homogenates of variants A, B and C were centrifuged and the pellets were suspended in an aqueous methylene blue solution (0.2%). With this staining method it is possible to discriminate by light microscopy between the strongly stained denatured but mechanically non-disrupted cells and empty cell wall envelopes obtained by mechanical cell disruption. Almost all cells were intact in variant A, whereas in variant C all cells were disrupted. In variant B about 60% of the cells were disrupted.

The result shows, that nucleic acids of high molecular weight do not permeate through the intact cell wall. RNA-molecules of the size known for t-RNA, however can be extracted with aqueous buffers from cells that were denatured with ethanol without mechanical cell wall disruption, since they can diffuse through the intact wall. After preservation of the nucleic acids in a hydrophilic volatile organic liquid according to the present invention it is therefore possible to use the cell wall as ultra-filter for selective extraction of a low-molecular-weight RNA. This may be favourable for the investigation of small RNA molecules like siRNA, miRNA and tnRNA.

### Example 3

1.5 ml of a mixture of ethanol (80% vol/vol) and a buffer (pH 3) prepared from citric acid (100 mM) and sodium dithionite (0.5%) were filled into Eppendorf tubes used for sample preservation and cell disruption. Small pieces (200 mg) of leafs of different plants (Arabidopsis thaliana, Daucus carota, Apium graveolens, Ceratophyllum demersum, Dactylorhiza incarnata) as well as flowers of Arabidopsis thaliana, Daucus carota and Apium graveolens and segments of roots and fruits of Arabidopsis thaliana were dispersed into this mixture. The amount of the tissue in the sample was estimated by the increase in the liquid level in the Eppendorf tube used for sample fixation.

The liquid mixture was sucked off after one hour and removed from the plant tissue pieces by pressing dry cellulose tissue with tweezers to the plant tissue material. Subsequently the plant tissue materials were dispersed in 1.5 ml of absolute ethanol. At the next day the ethanol medium was replaced by 1 ml fresh absolute ethanol and the dehydration strip described in example 2 was added. The tubes with the dehydration strip were shaken gently on rotating shaker until the next day. Subsequently the dehydration strips were removed and 200 mg of the fine glass beads (diameter 200 to 500 μm) and 10 large glass beads (diameter 3 mm) were added to each tube with the meanwhile decoloured leaf samples. The tubes were shaken for 20 min on a shaker for cell disruption (vibration mill MM2, Retsch GmbH, Germany) at 50% of the maximum intensity in horizontal position.

The samples were completely homogenised after this procedure, showing only ruptured cells and cell debris in the homogenate, when investigated by microscope. Small pieces of aggregating cells were only seen in the xylem vessels and the epidermis. Here, also all cells were opened. Comparison samples of fresh plant tissue immersed in water appeared either unchanged or showed very incomplete rupture of the tissue after shaking with the same intensity and duration with the glass beads.

Samples of 0.1 ml of the homogenates prepared in dehydrated ethanol were centrifuged. The obtained pellets were dissolved in 0.1 ml of an extraction buffer suitable for DNA and RNA extraction (1.4% SDS, 50 mM EDTA-Na, pH 7.5) and incubated over night at room temperature. 10 μl of the extracts were analysed by electrophoresis as described in example 2. Electrophoresis yielded a sharp DNA band, two rRNA bands responding to 28 S and 18 S rRNA as well as a broad band in the size region of tRNA in all cases. The good quality of the extracted high molecular nucleic acids was documented by the higher amount of rRNA found in the 28 S band compared to the smaller 18 S band. The procedure according to the invention enables also in case of plant tissue materials favourably high stability of high molecular nucleic acids during the extraction process in the aqueous extraction buffer. The same result was also obtained, when the ethanolic homogenates of the biological samples were stored for 4 weeks at room temperature.

1. A procedure for sample conservation and cell disruption, before extraction of nucleic acids in an aqueous extraction buffer, comprising:
introduction and equilibration of a biological sample containing a living cell, cell aggregate or tissue particle into a volatile organic liquid, that is miscible with water and that has a mass is in excess of a mass of the biological sample.

2. A procedure according to claim 1, wherein the biological sample, subsequently to equilibration with an almost water free organic liquid or after prolonged storage therein, is disrupted and homogenised by mechanical impulses.

3. A procedure according to claim 2, wherein cell disruption comprises:
shaking the sample in a dehydrated organic liquid with rigid disruption bodies, with a few large and heavy disruption beads and numerous small disruption beads.

4. A procedure according to claim 1, wherein the organic liquid surrounding the biological sample comes in contact with a water binding solid before mechanical disruption, with a zeolite with pores of 0.3 nm suitable for dehydration of organic liquids or with anhydrous crystals such as of sodium- or magnesium sulphate which are in a sufficient amount to ensure binding of residual water contained in a conservation vessel.

5. A procedure according to claim 1, wherein the living biological sample is denatured in a mixture of an organic liquid miscible with water and an aqueous buffer before transfer to an almost water-free volatile organic liquid, whereby a pH of the aqueous buffer is between 2 and 4 and its portion in the mixture is between 5 and 30%.

6. A procedure according to claim 5, wherein the applied mixture of an organic liquid and water contains mercaptoethanol, dithionite- or sulphite ions or a comparable reducing substance.

7. Preparation kit to conserve and prepare biological samples for extraction of nucleic acids for sample conservation and cell disruption, before extraction of nucleic acids in an aqueous extraction buffer, that includes introduction and equilibration of a biological sample containing a living cell, cell aggregate or tissue particle into a volatile organic liquid, that is miscible with water and that has a mass is in excess of a mass of the biological sample, wherein the preparation kit comprises:
an amount of a known substance suitable for dehydration of hydrophilic organic liquids with a pore size of 0.3 nm, which is adapted for every sample to a closable conservation and digestion vessel.

8. Preparation kit according to claim 7, which contains one dehydration body comprising:
the substance suitable for dehydration of hydrophilic organic liquids, the body being of a shape and size adapted to the vessel scheduled for sample preparation and stored in a water-vapor-tight envelope.

9. Preparation kit according to claim 7, with a device for mechanical cell disruption that is adapted in size and shape to the vessel scheduled for sample preparation, the cell disruption device being a mixture of cell disruption beads comprising a few large beads with a diameter above 2 mm and numerous small beads with a diameter below 0.5 mm.

10. Preparation kit according to claim 7, with a liquid mixture comprising:
a volatile organic liquid miscible with water and having a portion in the liquid of 70 to 95% (vol/vol) and an aqueous buffer with a pH between 2 and 4 having a portion in the mixture of 5 to 30%.

11. A procedure according to claim 2, wherein an organic liquid surrounding the biological sample comes in contact with a water binding solid before mechanical disruption, with a zeolite with pores of 0.3 nm suitable for dehydration of organic liquids or with anhydrous crystals which are in a sufficient amount to ensure binding of residual water contained in a conservation vessel.

12. A procedure according to claim 3, wherein an organic liquid surrounding the biological sample comes in contact with a water binding solid before mechanical disruption, with a zeolite with pores of 0.3 nm suitable for dehydration of organic liquids or with anhydrous crystals which are in a sufficient amount to ensure binding of residual water contained in a conservation vessel.

13. Preparation kit according to claim 8, with a liquid mixture comprising:
a volatile organic liquid miscible with water and having a portion in the liquid of 70 to 95% (vol/vol) and an aqueous buffer with a pH between 2 and 4 having a portion in the mixture of 5 to 30%.

14. Preparation kit according to claim 9, with a liquid mixture comprising:
a volatile organic liquid miscible with water and having a portion in the liquid of 70 to 95% (vol/vol) and an aqueous buffer with a pH between 2 and 4 having a portion in the mixture of 5 to 30%.

15. Preparation kit according to claim 10, containing a reducing substance such as sulphite, dithionite, or mercaptoethanol.

16. Preparation kit according to claim 13, containing a reducing substance such as sulphite, dithionite, or mercaptoethanol.

17. A procedure for preparation of biological samples containing living cells, cell aggregates or tissue particles for extraction of nucleic acids comprising:
introducing a biological sample into a volatile organic liquid that is miscible with water;
subsequent to equilibration with the liquid or after prolonged storage therein, dehydrating the sample by extraction of residual water with an almost dehydrated organic liquid or by its binding to a water binding solid;
disrupting the dehydrated sample in the dehydrated organic liquid phase by mechanical impulses; and
separating cell debris containing nucleic acids from the volatile organic liquid.

18. A procedure according to claim 17, wherein the disrupting is carried out by shaking the sample in the dehydrated organic liquid with rigid disruption bodies.

19. Process according to claim 17, wherein the biological sample is denatured in a mixture of an organic liquid miscible with water and an aqueous buffer before its transfer to an almost water-free volatile organic liquid, whereby a pH of the aqueous buffer is between 2 and 4 and its portion in the mixture is between 5 and 30%.

20. Process according to claim 19, wherein the mixture of an organic liquid and water contains mercaptoethanol, dithionite- or sulphite ions or a comparable reducing substance.
